(19) United States
(12) Patent Application Publication
(10) Pub. No.: US 2023/0384335 A1
(43) Pub. Date: Nov. 30, 2023

(54) APPARATUS AND METHOD FOR ACQUIRING AND ANALYZING BIOLOGICAL SAMPLES

(71) Applicant: GOTTARDO ADVISORY LIMITED, Hong Kong (CN)

(72) Inventors: David Lyle SCHNEIDER, Hong Kong (CN); Tyvin A. RICH, Hampton, VA (US); Martin A. ATWOOD, Brooklyn, NY (US)

(21) Appl. No.: 18/296,384

(22) Filed: Apr. 6, 2023

Related U.S. Application Data
(63) Continuation of application No. PCT/IB2021/059155, filed on Oct. 6, 2021.

(30) Foreign Application Priority Data
Oct. 7, 2020 (US) 63088526

(51) Int. Cl. G01N 35/00; G16H 10/40; G16H 15/00; G16H 40/67

(52) U.S. Cl. CPC G01N 35/00732; G16H 10/40; G16H 15/00; G16H 40/67; G01N 2035/00891; G01N 2035/00831

(57) ABSTRACT

Apparatus and method for acquiring and analyzing biological samples are provided. The apparatus may be in the form of a free-standing apparatus, and include: means for receipt of identifying data of the individual; means for receiving the biological sample for the individual; means for providing a unique identifier for the biological sample and association with the individual; and means for conducting an analysis of the biological sample and generating a report of analysis results; wherein all of the foregoing means are under control of a microprocessor located in the apparatus. The apparatus and method allow fast processing of samples.

### CROSS-REFERENCE TO RELATED APPLICATION

This application claims the benefit of U.S. application No. 63/088,526, filed on Oct. 7, 2020. The entire disclosure of the above-referenced application is incorporated herein by reference in entirety for all purpose.

### TECHNICAL FIELD

The present disclosure is directed to a detection kiosk that provides fast and accurate pathogen identification obtained from human samples to allow for rapid results. Such identification information can be used in a variety of environments for public health safety decision-making about the subject being tested. The detection kiosk can be linked to public health databases and, through adaptive learning such databases can analyze the received information to provide immediate enhanced test results that can be used, for example, at airport and border security locations so that processing times for such populations are not disrupted. The kiosk system addresses the need for enhanced, rapid, and accurate health screening.

### BACKGROUND ART

Medical imaging technology and diagnostic systems continue to improve with the goal of providing health-safety solutions to the public. The public health benefits derived from these technical and analytical software advancements are very beneficial, potentially saving significant amounts of time and money, and can add efficiencies to large scale examination and testing protocols. However, along with these advancements have come additional challenges, including the reliability of results, the speed of reporting and the uniformity of the created data, all of which are exacerbated by the complexity and the degree of inter-connection among the public safety, medical, and health-care industry systems.

The pandemic of 2020 has brought to the fore the question of how to protect the health of nations without restricting free movement of healthy individuals, but with the security and surety of a uniform, rapid and accurate testing and reporting system. The collection of “good data” has been cited as a matter of concern within the medical community; the quality of collected data is of primary importance in making informed decisions about public health policy and border security.

Current industry solutions tend to rely on conventional methods that typically employ imperfect testing kits and laboratory analysis, where the results can often be incorrect in as high as 60% of the test results. A Rutgers University press release of May 2020 repots that saliva tests are being touted as a solution for mass screenings.

The Gold Standard test for SARS viruses is not a rapid test and requires a laboratory to carry testing out. It is a highly technical process that requires the right machine, facilities and a proper safety infrastructure, such as may be found in a biosafety-level laboratory. An alternative method disease diagnosis and screening, using imaging for a rapid diagnosis, led by Pratik Shah et al of MIT and reported in an online press release in 2020, applies adaptive learning algorithms for medical imaging. Adaptive learning and image capture are being used with much success in China and the United States in the radiology, pathology, and ophthalmology fields, among others.

Test kits for pathogens are only one step of a health passport concept, and they are cumbersome, can be contaminated, misused, or expired, and can create labor intensive situations, especially in high-volume environments, such as homeland security venues and for use in the travel and airline industries. The results must be manually entered into a database and recorded by TSA or other reliable personnel, other than the person being tested.

Current industry methods and standards for border security do not include solutions for the need of secure big data capture of virus and other pathogen screenings in real time and the logistics for mass screenings, especially when airports, border checkpoints and other portals are operating at high capacity.

Current Industry standards for virus analysis and screening are mostly limited to a specific virus test, using chemical analysis and/or imaging after sample preparation. Recent advancements with nanotechnology promise more rapid analysis and more rugged platforms for virus testing.

### SUMMARY OF INVENTION

An example embodiment provides a free-standing apparatus for acquisition and analysis of a biological sample from an individual. The apparatus includes: means for receipt of identifying data of the individual; means for receiving the biological sample for the individual; means for providing a unique identifier for the biological sample and association with the individual; and means for conducting an analysis of the biological sample and generating a report of analysis results; wherein all of the foregoing means are under control of a microprocessor located in the apparatus.

Another example embodiment provides a method for acquisition and analysis of a biological sample from an individual. The method includes operations of: receiving identifying data of the individual; receiving the biological sample for the individual; providing a unique identifier for the biological sample and association with the individual; and conducting an analysis of the biological sample and generating a report of analysis results.

### BRIEF DESCRIPTION OF DRAWINGS

Aspects of the present disclosure may be appreciated upon consideration of the following description of exemplary embodiments when taken in conjunction with the accompanying drawings.

FIG. 1 is a pictorial representation of an embodiment of a kiosk of the inventive mechanism.

FIG. 2 is a flow diagram illustrating the sample capture methodology.

FIG. 3 is an illustration of a capture media material that lends itself to multiple unit deployments in “media pods” or “media cartridges”.

FIG. 4 is an illustration of a capture material element queued for entry into a cartridge receiver.

FIG. 5 is a flow diagram of the capture and recording of chain-of-custody data for incorporation into embodiments that provide for evidence retention and recording.

FIG. 6 is a flow diagram of the inputting and recording of biometric identifiers for an operator of the kiosk and individual being tested.

FIG. 7 is a flow diagram of the methodology for the export of recorded data into large data sets for comparison, identification, reporting and research processing.

### DETAILED DESCRIPTION

The present disclosure is directed to a self-serve automated apparatus, including devices and kiosk embodiments, that can be employed in a variety of settings, such as airports, for testing individuals prior to immigration or boarding; in hospitals in conjunction with both patient admitting and visitor clearance; as well as in schools and other facilities requiring fast and accurate pathogen screenings for large numbers of people. Using adaptive learning and a remote database, the present disclosure can quickly analyze a sample and provide a real time report, allowing the individual being tested to receive an accurate and complete health check while the results of the test are stored in the database for subsequent authorized use and retrieval as may be needed.

The present disclosure provides an apparatus which may be in the form of a free-standing kiosk, allowing fast processing of samples. Such kiosks may be located in high traffic environments, such as airports or entertainment venues, where the analysis of many samples must be performed rapidly. The apparatus and method have high-assurance for tracking and indexing.

The present disclosure comprises a kiosk collection apparatus that is intended to provide onsite accurate pathogen testing by the analysis of human-derived samples, and includes means for identifying and documenting the individual to be tested; means for drawing samples pursuant to a collection protocol including sample verification; and on-site analysis of the sample. The analysis can be performed through microprocessor control at the kiosk or the raw data can be forwarded to an off-site recipient for analysis and return of results.

The kiosk may also include a self-contained cleaning system to sterilize the sample collection chamber in the kiosk after a sample is collected, permitting the next test to commence within minutes. Collection means for the sample, such as an internal bin, may be provided to collect the tested samples with safety, and may include appropriate waste sterilization compartments.

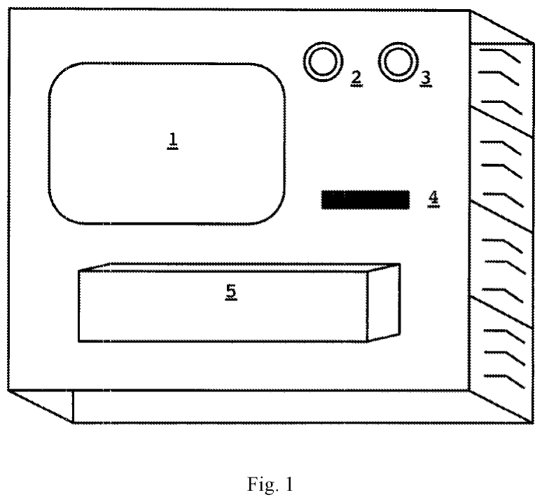

FIG. 1 presents one illustrative embodiment that provides the system for analysis of a specimen taken from an individual. As seen therein, the system is in the form of a kiosk that acquires a sample for analysis as well as identity validation data from the individual from whom the sample is taken. An authorized and trained operator of the kiosk manually takes the sample, such as by a swab, which is loaded into the kiosk through specimen intake drawer 5. The kiosk can deliver human interface instructions through a video display or touch-screen display 1. The kiosk acquires identification and validation information of the individual using biometric interfaces, which may be in plurality, such as including a video camera function via a video camera 2 and audio input and output functions via audio input and output 3. Identification cards, tokens or RFID are validated through card reader slot function via a card slot 4, as known in the art. The audio input and output 3 may be a speaker/microphone combination.

To prevent unauthorized operation of the kiosk, the operator is provided with an access or identification card, which must be inserted into the card slot 4 to operate the kiosk. This ensures that a test is performed only by an authorized agent. In alternate embodiments, the kiosk can integrate various commercially-available mechanisms for recording and confirming the identity of the agent. The agent can also be validated through use of the biometric interfaces.

In contemplated alternative embodiments, the system can establish the identify of an individual physically present in proximity to the kiosk by scanning the individual's passport or other official identification through a reader/scanner, either as a separate interface (not shown) or incorporating such functionality into the video camera 2. Further confirmation of the individual's identity as matching the individual on scanned document may be performed through other biometric characteristics. The kiosk can provide audio instructions by audio output and video instructions, prompted on touch-screen display 1, and can provide the individual with instructions as needed. All such functions may be under the control of the system kiosk's main microprocessor, suitably programmed as known in the art. The individual can also enter information through the touch-screen.

With the identity of the individual being confirmed, a microprocessor-controlled analysis system in the kiosk analyzes the sample. The analysis system may include a high definition microscopy component camera system with analytical software for imaging analysis and labeling. The kiosk bundles the analysis results with both the identification on the authorized agent and the individual tested and transmits the data to a remote electronic data storage facility. The data processing modules employed provide for encoding and privatization of all data. Such data processing modules may provide random research identifiers and encode private individual data into private electronic data storage encrypted at multiple levels. In some instances, the data delivered into remote data storage, which may be cloud-based, can be de-identified and privatized so that the results of the analysis, but not the individual associated with the results, is stored. This may allow bulk analysis of results, such as to measure trends and identify potential “hot spot” areas, without endangering the privacy of the tested individuals. After the analysis at the kiosk and transmission of the data to the remote server(s), the data is automatically purged from the kiosk's memory; it never remains on the kiosk device.

Depending on the nature of the pathogen to be identified and the sophistication of the analysis software, the analysis may be conducted at the kiosk under microprocessor control, with communication to remote databases as needed. Alternatively, the raw test data may be transmitted to a remote facility for analysis, with the results transmitted back to the kiosk. In either case, upon completion of the analysis a report is provided to the kiosk operator and, if proper, shared with the individual tested. Depending on the results, the individual may be cleared for passage or otherwise advised or directed to seek treatment or other processing.

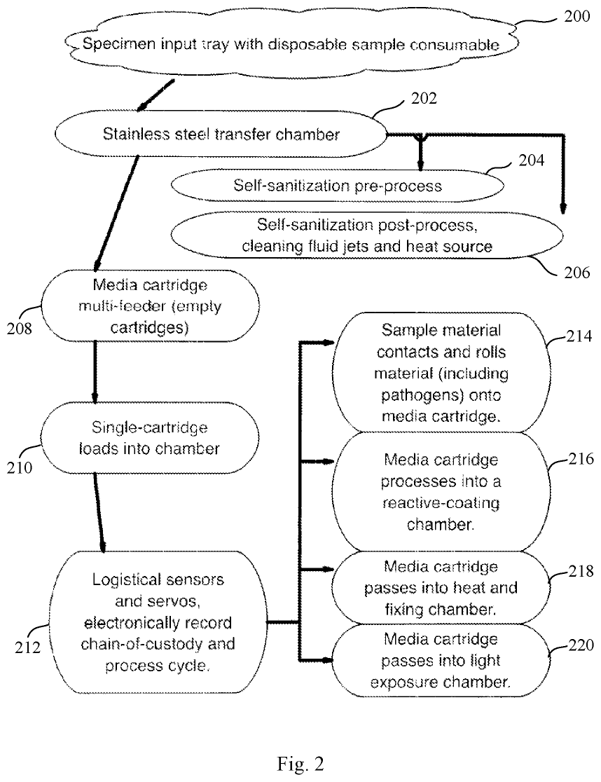

FIG. 2 illustrates the procedures in the acquisition and recording process, setting forth components that may be utilized for the procedures. The physical mechanism may use sealed chambers, enclosed in polished stainless steel or other metal, polymer or other synthetic material encasements, as known. The encasements provide functionality for self-sanitation, such as by use of an autoclave process, which in turn may include one or more of chemical, radio-frequency, and UV options. It is further contemplated that embodiments may use radio spectrum radiation or radioisotopes for different levels of sterilization inside an appropriate encasement.

As presented in FIG. 2, the sample specimen is received on an input tray at 200. The tray is moved into a transfer chamber at 202. The chamber is prepared for receipt of the sample by incurring a pre-process sanitation at 204. Such process may include the use of appropriate cleaning and sanitizing fluids as well as heat, as shown at 206.

The actual samples to be analyzed may be received by individual cartridges arrayed in a multi-cartridge feeder or carrier at 208. A single cartridge receives a specific sample, and delivers it into the sterilized transfer chamber at 210. An electronic record is created of the sample identification and other information to create an appropriate chain-of-custody record at 212.

The sample material received from the individual is placed on a media cartridge at 214. The cartridge subsequently passes into a plurality of chambers, where it is exposed to various fixing and analysis procedures as appropriate to the analysis desired illustrated at 216-220. Such procedures may also include sample sterilization and de-contamination.

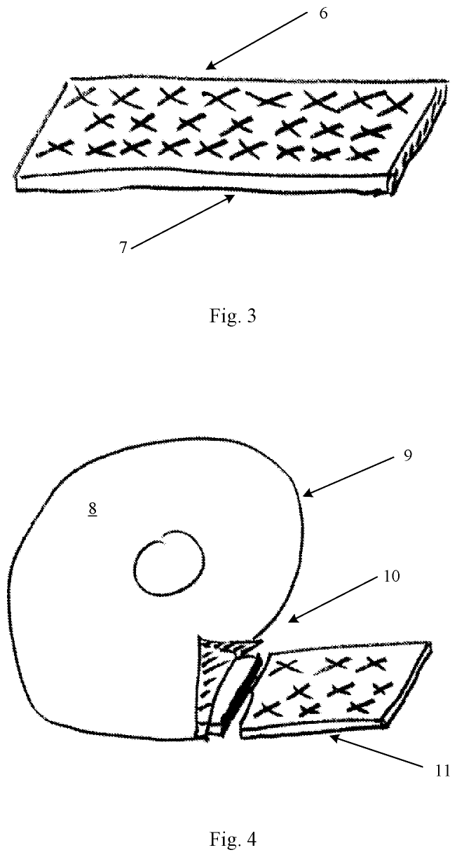

FIG. 3 illustrates an embodiment of a flexible, translucent, mesh-type material 6 which can support the receipt of various of reactive-agent chemical and biological-reactive materials as may be required for sample analysis. The porous mesh material 6 is attached to a rider material 7 that may be non-porous, flexible, and appropriate for visible light photography. For example, the material may be a rubber formulation or more specifically a synthetic plastic/petroleum composite formulated to meet different temperature and light requirements as appropriate. The mesh structure may contain reactive materials for the pathogen to be analyzed, with micro- and/or nano-structures, allowing the apparatus to records the reactions with a microscopic photographic process.

Other techniques for analysis of the specimen on the capture material may include fluorescent photographic electronic sensors, optionally with magnifying and filtering lenses. Such sensors can record colors, heat, intensity, and size variations produced by the chemical reactions within the media substrate material, providing additional data to assist the sample analysis.

The specimen capture media may advantageously be of a translucent media to facilitate the acquisition of microphotographic recordings, allowing multiple captures are performed as an analysis is performed. Such captures may employ variations in the applied light spectrum to capture unique multiple chromatic recordings of the media. As may be appropriate, temperature adjustment may be performed while recording, whereby the temperature variations are recorded and combined with resulting visual recording.

FIG. 4 illustrates a media material dispensed from a continuous-web feed mechanism. This media cartridge, which may be of a barrel-construction 8, is loaded with a roll of media 9 which are dispensed or fed out from the barrel-construction 8 through a conveyor feed mechanism 10. The feed mechanism applies the required reactive and activation materials onto the individual mesh media and provides cutting and separation into individual sample-receiving elements. The individual media elements are carried by a chamber conveyor 11 into a sterile chamber for receipt of a sample, sample analysis and subsequent destruction.

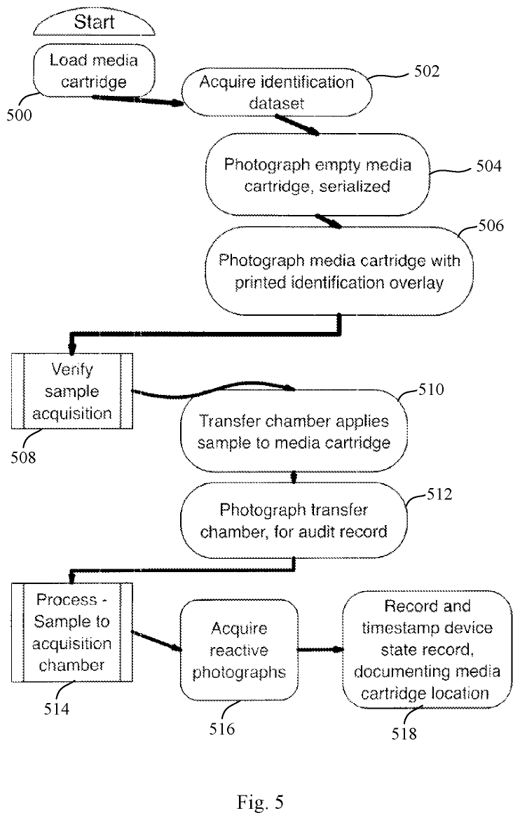

FIG. 5 presents the procedure for the chain-of-custody and evidentiary functions. The key functionality performed by the system components is that each individual media unit is identified and recorded with individual and tracked serialization. This serialization is validated prior to specimen processing and recorded for official record in final processing.

As illustrated, a media cartridge is loaded for sample receipt at 500. It is assigned an individual identifier at 502 and a reference photograph of the cartridge is taken at 504 and associated with the identification data set assigned at 506. The sample to be received by the cartridge is received and verified at 508, and is transferred to the cartridge at 510. A photographic record of the transfer cartridge is created for audit tracking and recording at 512.

The cartridge with sample then proceeds to an acquisition chamber for commencement of the analysis procedure at 514, during which analysis photographic and other records are created to document the analysis at 516, along with a timestamp and other data to document the cartridge location and analysis performed at 518.

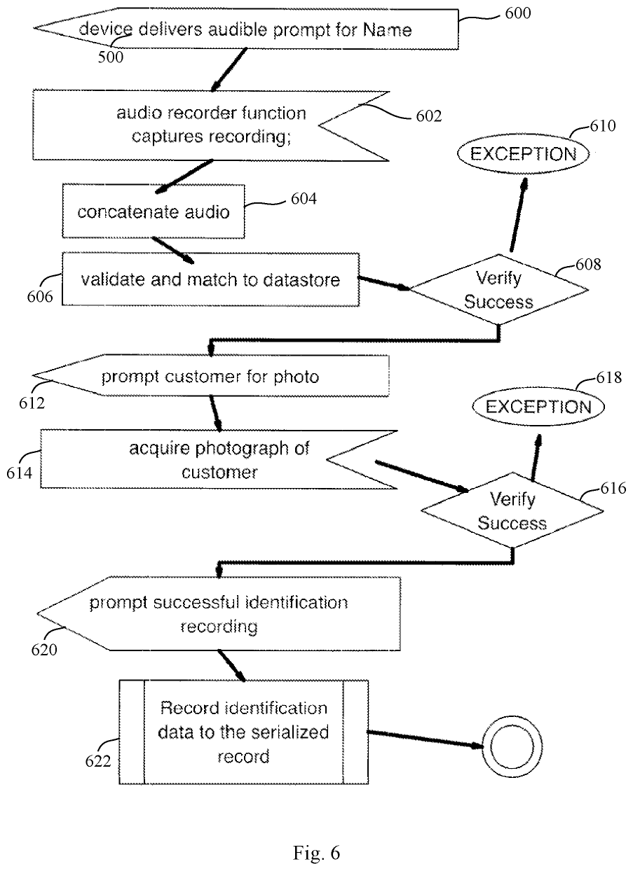

FIG. 6 describes a method and process for establishing and capturing identification information of an individual from whom a sample is to be taken. As shown therein, the system establishes contact with the individual by issuing an audible (or other) prompt for the individual's name at 600. The response is captured at 602, concatenated with other data at 604, and then used to initially validate the identity of the individual at 606. If the validation fails at 608, an exception is raised at 610, and further processing is ended. The individual can then discuss the situation with the kiosk operator, perhaps with a repeat of the validation process.

Elf the validation is successful the individual is prompted for a face photograph at 612, and the photo is taken and acquired by the system at 614. Again, the photo is compared to another photograph of the individual, such as a passport photo, and with verification success confirmed at 616, recording of the identification procedure is authorized at 620 and recordation performed at 622, with the information added to the serialized record for the media cartridge to be used for sample collection. If the photographic validation fails, an exception issues at 618 for re-test or other appropriate action.

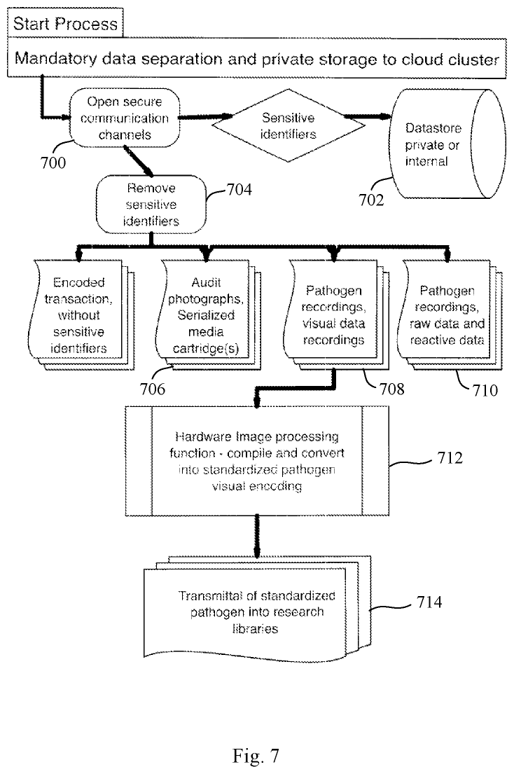

FIG. 7 illustrates the procedure for processing and storing data associated with a sample analysis. As shown therein, secure communication channels are established at 700 and a full record, including material characterized by a sensitive identifiers is transmitted to a primary secure data store at 702. To allow transmission of data to other facilities that do not require access to a full record, including “sensitive identifiers” in the record, such identifiers are scrubbed from the record at 704. Such identifiers may include the tested individuals name, personal information, passport or other identification documents, and the like. The remaining “cleansed” record may continue to contain data directly associated with the test or analysis performed, such as audit photograph and serialized media cartridge identification 706, information directly related to the pathogen(s) tested for and identified 708, 710. The cleansed record is then compiled and processed into a desired standard form to enable entry into data banks and cross-referencing at 712. With such processing completed the record is delivered at 714 to the appropriate recipients, such as a research library or institution.

In some situations, some sensitive identifiers may remain in a scrubbed record, depending on the nature of the recipient for the information. For example, medical or hospital personnel having a relationship with the individual tested may be granted access by the individual to receive the results of a test for entry into the individual's personal medical record. Information regarding such authorized access by be provided by the individual during an initial “interview” when the individual's identity is entered into and recorded by the kiosk.

The present system allows integration of sample analysis and data collection by a kiosk or other testing station to be both maintained by the kiosk as well as passed to other facilities for review and correlation with data for the tested individual obtained from other authorized sources, as well as with a virus library or libraries with adaptive learning capabilities such that an accurate result of the test can be determined and the libraries themselves can be updated with the results of the test. The results of the analysis can is transmitted back to the kiosk for use by the agent and individual being tested.

With adaptive learning and predictive analysis matched against the libraries, the kiosk can differentiate among a negative pathogen sample test; a positive pathogen sample test; and an unknown pathogen sample. The latter two results can provide grounds for further examinations and potential refusal of passage and isolation.

In addition to use at airports and similar travel locations and hospitals, the kiosk system can be used in a variety of venues where a large number of people needed to be checked quickly and accurately, such as sports venues and large social gatherings.

Similar to the above free-standing apparatus for acquisition and analysis of a biological sample from an individual, the present disclosure also provides a method for acquisition and analysis of a biological sample from an individual. The method may include operations of: receiving identifying data of the individual; receiving the biological sample for the individual; providing a unique identifier for the biological sample and association with the individual; and conducting an analysis of the biological sample and generating a report of analysis results.

In some embodiment, all of the foregoing operations are under control of a microprocessor located in a free-standing apparatus.

In some embodiment, the operation of receiving identifying data uses at least one of a camera, a scanner, a monitor and a speaker/microphone combination.

In some embodiment, the method further includes an operation of verifying the identity of an operator so that the operator can control of the free-standing apparatus with respect to an acquisition of the biological sample.

In some embodiment, the operation of receiving identifying data uses: an interface for prompting name and identification information of the individual; with user interfaces in plurality; verbal guidance means for general instructions to the individual; and means for visual guidance through a monitor.

In some embodiment, the method further includes an operation of storing the analysis report, whereby each report receives an individual and identification number.

In some embodiment, the method further includes an operation of sanitizing the biological sample after analysis.

In some embodiment, the method further includes an operation of editing the report to delete specifically-identified information, and an operation of transmitting both the report and the edited report to remote facilities, either for analysis or storage.

In some embodiment, the operation of conducting an analysis uses components located at least in the free-standing apparatus or at a remote location.

In some embodiment, the operation of receiving identifying data includes: establishing contact with the individual by issuing a prompt for the individual's name; capturing the individual's response; concatenating with a datastore to validate the identity of the individual; prompting the individual for a face photograph to take and acquire the photograph; comparing the photograph to another photograph of the individual; authorizing recording of the identification procedure and performing recordation; and adding information to a serialized record for a media cartridge to be used for sample collection.

In some embodiment, the operation of receiving the biological sample includes: receiving a sample specimen on an input tray; moving the input tray into a transfer chamber; receiving actual samples to be analyzed by individual cartridges arrayed in a multi-cartridge feeder, wherein a single cartridge receives a specific sample, and delivers it into a sterilized transfer chamber; creating an electronic record of the sample identification and other information to create an appropriate chain-of-custody record; placing sample material received from the individual on a media cartridge, wherein the media cartridge subsequently passes into a plurality of chambers, where it is exposed to various fixing and analysis procedures as appropriate to the analysis desired.

In some embodiment, the operation of providing a unique identifier includes: loading a media cartridge for the sample; assigning an individual identifier or the media cartridge; taking a reference photograph of the media cartridge; associating the reference photograph with the identification data set; transferring the sample to the media cartridge; creating a photographic record of the transfer cartridge for audit tracking and recording; and proceeding the cartridge with sample to an acquisition chamber for commencement of the operation of conducting an analysis, during which analysis photographic and other records are created to document the analysis, along with a timestamp and other data to document the cartridge location and analysis.

1. A free-standing apparatus for acquisition and analysis of a biological sample from an individual, comprising:
means for receipt of identifying data of the individual;
means for receiving the biological sample for the individual;
means for providing a unique identifier for the biological sample and association with the individual; and
means for conducting an analysis of the biological sample and generating a report of analysis results;
wherein all of the foregoing means are under control of a microprocessor located in the apparatus.

2. The apparatus of claim 1, wherein the means for receipt of identifying data comprises at least one of a camera, a scanner, a monitor and a speaker/microphone combination.

3. The apparatus of claim 1, further comprising means for operator control of the apparatus with respect to an acquisition of the biological sample, the means for operator control comprising means for verifying the identity of the operator.

4. The apparatus of claim 1, wherein the means for receipt of identifying data comprises:
an interface for prompting name and identification information of the individual; with user interfaces in plurality;
verbal guidance means for general instructions to the individual; and
means for visual guidance through a monitor.

5. The apparatus of claim 1, further comprising means for storage of the analysis report, whereby each report receives an individual and identification number.

6. The apparatus of claim 1, further comprising means for sanitizing the biological sample after analysis.

7. The apparatus of claim 5, further comprising means for editing the report to delete specifically-identified information, and means for transmitting both the report and the edited report to remote facilities, either for analysis or storage.

8. The apparatus of claim 1, wherein the means for conducting an analysis comprises components located at least in the apparatus or at a remote location.

9. A method for acquisition and analysis of a biological sample from an individual, comprising operations of:
receiving identifying data of the individual;
receiving the biological sample for the individual;
providing a unique identifier for the biological sample and association with the individual; and
conducting an analysis of the biological sample and generating a report of analysis results.

10. The method of claim 9, wherein all of the foregoing operations are under control of a microprocessor located in a free-standing apparatus.

11. The method of claim 9, wherein the operation of receiving identifying data uses at least one of a camera, a scanner, a monitor and a speaker/microphone combination.

12. The method of claim 10, further comprising an operation of verifying the identity of an operator so that the operator can control of the free-standing apparatus with respect to an acquisition of the biological sample.

13. The method of claim 9, wherein the operation of receiving identifying data uses:
an interface for prompting name and identification information of the individual; with user interfaces in plurality;
verbal guidance means for general instructions to the individual; and
means for visual guidance through a monitor.

14. The method of claim 9, further comprising an operation of storing the analysis report, whereby each report receives an individual and identification number.

15. The method of claim 9, further comprising an operation of sanitizing the biological sample after analysis.

16. The method of claim 14, further comprising an operation of editing the report to delete specifically-identified information, and an operation of transmitting both the report and the edited report to remote facilities, either for analysis or storage.

17. The method of claim 10, wherein the operation of conducting an analysis uses components located at least in the free-standing apparatus or at a remote location.

18. The method of claim 9, wherein the operation of receiving identifying data comprises:
establishing contact with the individual by issuing a prompt for the individual's name;
capturing the individual's response;
concatenating with a datastore to validate the identity of the individual;
prompting the individual for a face photograph to take and acquire the photograph;
comparing the photograph to another photograph of the individual;
authorizing recording of the identification procedure and performing recordation; and
adding information to a serialized record for a media cartridge to be used for sample collection.

19. The method of claim 9, wherein the operation of receiving the biological sample comprises:
receiving a sample specimen on an input tray;
moving the input tray into a transfer chamber;
receiving actual samples to be analyzed by individual cartridges arrayed in a multi-cartridge feeder, wherein a single cartridge receives a specific sample, and delivers it into a sterilized transfer chamber;
creating an electronic record of the sample identification and other information to create an appropriate chain-of-custody record; and
placing sample material received from the individual on a media cartridge, wherein the media cartridge subsequently passes into a plurality of chambers, where it is exposed to various fixing and analysis procedures as appropriate to the analysis desired.

20. The method of claim 9, wherein the operation of providing a unique identifier comprises:
loading a media cartridge for the sample;
assigning an individual identifier or the media cartridge;
taking a reference photograph of the media cartridge;
associating the reference photograph with the identification data set;
transferring the sample to the media cartridge;
creating a photographic record of the transfer cartridge for audit tracking and recording; and
proceeding the cartridge with sample to an acquisition chamber for commencement of the operation of conducting an analysis, during which analysis photographic and other records are created to document the analysis, along with a timestamp and other data to document the cartridge location and analysis.
